(19) United States
(12) Patent Application Publication
(10) Pub. No.: US 2025/0084082 A1
(43) Pub. Date: Mar. 13, 2025

(54) HUPERZINE B CRYSTAL AND PREPARATION AND APPLICATION THEREOF

(71) Applicant: SHANGHAI TIN TSZ BIO VALLEY BIOLOGICAL ENGINEERING CO., LTD., Shanghai (CN)

(72) Inventors: Delong XIE, Shanghai (CN); Wangzhou CHI, Shanghai (CN); Yonggang LI, Shanghai (CN); Xuhua HU, Shanghai (CN); Bo GU, Shanghai (CN); Dongyan SUN, Shanghai (CN); Zhiqiang LIU, Shanghai (CN); Baosheng YIN, Shanghai (CN)

(21) Appl. No.: 18/579,816

(22) PCT Filed: Jul. 13, 2022
(86) PCT No.: PCT/CN2022/105559

§ 371 Date: Nov. 25, 2024

(30) Foreign Application Priority Data
Jul. 13, 2021 (CN) 202110787427.0

(51) Int. Cl. C07D 471/08

(52) U.S. Cl. CPC C07D 471/08

(57) ABSTRACT

The present invention provides a huperzine B crystal and a preparation thereof. Specifically, the present invention provides a crystal form A of the huperzine B crystal. An X-ray diffraction pattern of the crystal form A has the following 2θ angular characteristic absorption peak: 9.56±0.2, 13.90±0.2, 14.88±0.2, 16.00±0.2, 25.39±0.2, and 28.78±0.2.

### TECHNICAL FIELD

The present invention belongs to the field of pharmaceutical technology, and specifically relates to a crystal form of HuperzineB, and the preparation therefor.

### BACKGROUND

Alzheimer's disease, also known as senile dementia, is a neurodegenerative disease clinically characterized by progressive memory and cognitive deficits with multiple etiological factors. The incidence increases with age. Degeneration of the cerebral cortex of patients leads to the loss of normal activity functions, including memory and judgment decline, lack of reasoning ability and slowed behavior. The loss of the ability to live independently brings a heavy mental and financial burden on the family.

Huperziaserrata (Thunb.) Trev. (also known as huperzia and lycopodium serratumthunb.) is a plant that used as a medicinal plant. Huperzine B, an alkaloid extracted from Huperziaserrata, is a cholinesterase inhibitor with the advantages of low toxicity, high potency, reversibility and high selectivity. Cholinesterase inhibitor is one of the few effective drugs currently approved by the FDA for the treatment of Alzheimer's disease. There are currently no pharmaceutical formulations of Huperzine B as a single component available on the market.

The structural formula of Huperzine B is as follows:

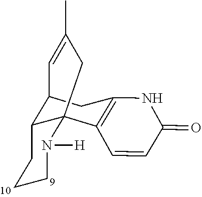 [SMILES: CC1=CC2Cc3[nH]c(=O)ccc3C3(C1)NCCCC23]

The chemical name of Huperzine B is: (4aR, 5R, 10bR)-1,2,3,4,4a,4,6,10b octahydro-12-methyl-5,10b-propeno-1,7-phenanthrolin-8 (7H)-one.

Compared to Huperzine A, Huperzine B has a certain degree of toxicity-reducing and synergistic effect. In addition, Huperzine B has the functions of inhibiting cholinesterase activity and improving learning and memory efficiency. The strength of its action is not as strong as that of Huperzine A, but its duration of action is longer than that of Huperzine A and its adverse effects are fewer than those of Huperzine A. In addition, it has certain neuroprotective effect. When used in combination with Huperzine A, it can reduce the dosage and frequency of Huperzine A, and reduce adverse effects. Therefore, Huperzine B has medicinal value worth developing and is expected to be a clinically applicable drug. However, there is a lack of HuperzineBcrystal forms suitable for pharmaceutical use in the field.

### SUMMARY

After extensive research, the inventor of the present invention has discovered a crystal form A of Huperzine B, which has the advantages of excellent physicochemical properties and good stability, and can meet the requirements of medicinal preparations on crystalline Huperzine B, and is suitable for industrialized production.

A first purpose of the present invention is to fill the gap in the prior art and provide a crystal form A of Huperzine B.

Another purpose of the present invention is to provide a preparation method for the crystal form A of Huperzine B.

In a first aspect of the present invention, provided is a crystal form A of Huperzine B, wherein the X-ray diffraction pattern of the crystal form A has characteristic absorption peaks at the following 2θ angular: 9.56±0.2, 13.90±0.2, 14.88±0.2, 16.00±0.2, 25.39±0.2 and 28.78±0.2.

In another preferred embodiment, the X-ray diffraction pattern of the crystal form further has one or more characteristic absorption peaks at 2θ angular selected from the group consisting of: 12.08±0.2, 13.26±0.2, 14.12±0.2, 15.18=0.2, 16.86±0.2, 18.45±0.2, 19.12±0.2, 19.84±0.2, 22.41±0.2, 22.97±0.2, 23.42±0.2, 24.79±0.2, 25.81±0.2, 26.01±0.2, 26.34±0.2, 27.65±0.2, 28.04±0.2, 28.44±0.2, 29.86±0.2, 30.51±0.2, 31.22±0.2, 33.22±0.2, 34.94±0.2, 37.09±0.2 and 38.64±0.2.

In another preferred embodiment, the infrared spectrum of the crystal form as measured by KBr compression has one or more characteristic absorption peaks selected from the group consisting of: 3323.36 cm⁻¹, 3219.52 cm⁻¹, 3088.83 cm⁻¹, 2994.76 cm⁻¹, 2958.33 cm⁻¹, 2938.33 cm⁻¹, 2913.38 cm⁻¹, 2861.67 cm⁻¹, 2817.87 cm⁻¹, 1666.30 cm⁻¹, 1605.50 cm⁻¹, 1555.70 cm⁻¹, 1453.09 cm⁻¹, 1429.96 cm⁻¹, 1369.99 cm⁻¹, 1346.50 cm⁻¹, 1323.03 cm⁻¹, 1309.51 cm⁻¹, 1283.99 cm⁻¹, 1260.62 cm⁻¹, 1200.47 cm⁻¹, 1180.20 cm⁻¹, 1155.23 cm⁻¹, 1127.13 cm⁻¹, 1099.03 cm⁻¹, 1071.87 cm⁻¹, 1055.99 cm⁻¹, 1046.15 cm⁻¹, 993.81 cm⁻¹, 973.82 cm⁻¹, 958.22 cm⁻¹, 915.72 cm⁻¹, 867.03 cm⁻¹, 855.87 cm⁻¹, 839.87 cm⁻¹, 815.10 cm⁻¹, 783.02 cm⁻¹, 755.87 cm⁻¹, 731.69 cm⁻¹, 686.04 cm⁻¹, 651.90 cm⁻¹, 629.38 cm⁻¹, 568.32 cm⁻¹, 521.58 cm⁻¹, 499.42 cm⁻¹, 487.62 cm⁻¹, 467.39 cm⁻¹, 442.58 cm⁻¹ and 417.99 cm⁻¹.

In another preferred embodiment, the Raman spectrum of the crystal form has one or more characteristic absorption peaks selected from the group consisting of: the absorption peaks were found at 2939.42 cm⁻¹, 2909.62 cm⁻¹, 2879.72 cm⁻¹, 2864.57 cm⁻¹, 2820.5 cm⁻¹, 1674.43 cm⁻¹, 1602.88 cm⁻¹, 1553.75 cm⁻¹, 1450.8 cm⁻¹, 1288.9 cm⁻¹, 1262.33 cm⁻¹, 1246.66 cm⁻¹, 719.965 cm⁻1 and 683.93 cm⁻¹.

In a second aspect of the present invention, provided is a preparation method of the crystal form according to the first aspect of the present invention, including the steps of:
1) dissolving Huperzine B in a first solvent under heating conditions to obtain a first solution of Huperzine B;
2) performing the following step 2a) or 2b):
   2a) cooling down the first solution of Huperzine Band slowly evaporating the solvent to dryness;
   2b) cooling down to room temperature to precipitate crystals and keeping at about 0° C.;
3) drying the product obtained in step 2) in vacuum and collecting the resulting solid to obtain the crystal form.

In another preferred embodiment, when step 2a) is employed, in step 1), the first solvent is selected from the group consisting of: methanol, ethanol, n-butanol, 1,4-dioxane, 1,2-dichloroethane, dichloromethane, acetone, water, and combinations thereof.

In another preferred embodiment, when step 2a) is employed, in step 1), the ratio of solvent to Huperzine B is 1: 15-75 (ml: mg); and/or
in step 1), the heating temperature is 50° C.-90° C.

In another preferred embodiment, the first solvent is a mixture of a third solvent and a fourth solvent, and step 1) comprises: heating and dissolving completely in the third solvent, then adding the fourth solvent and dissolving completely under heating conditions.

In another preferred embodiment, when step 2b) is employed, the first solvent is selected from the group consisting of: ethanol, n-butanol, 1,4-dioxane, 1,2-dichloroethane, N,N-dimethylformamide, acetone, water, methyl tert-butyl ether, and combinations thereof.

In another preferred embodiment, when step 2b) is employed, in step 1),the ratio of solvent to Huperzine B is 1: 15-90 (ml: mg); and/or
in step 1), the heating temperature is 70° C.-90° C.

In a third aspect of the present invention, provided is a preparation method of the crystal form as described in the first aspect of the present invention, the method comprising the steps of:
i) adding Huperzine B to a second solvent, heating and stirring and keeping for 48-96 hours to obtain a reaction mixture;
ii) placing the reaction mixture in an open container and evaporating the solvent to dryness at room temperature;
iii) drying in vacuum and collecting the resulting solid to obtain the crystal form.

In another preferred embodiment, the second solvent is selected from the group consisting of toluene, ethyl acetate, methyl tert-butyl ether, and combinations thereof.

In another preferred example, in step i), the ratio of solvent to Huperzine B is 1: 2-10 (ml: mg); and/or
instep ii), the heating temperature is 40° C.-60° C.

It should be understood that within the scope of the present invention, the above-mentioned technical features of the present invention and the technical features specifically described in the following (such as examples) can be combined with each other to form a new or preferred technical solution. Limited by space, it will not be repeated here.

### DESCRIPTION OF THE DRAWINGS

FIG. 1 shows a typical X-ray powder diffraction pattern of the Huperzine B crystal form A of Example 1;

FIG. 2 is an X-ray powder diffraction pattern of the Huperzine B crystal form A of Example 2;

FIG. 3 is an X-ray powder diffraction pattern of the Huperzine B crystal form A of Example 3;

FIG. 4 is an X-ray powder diffraction pattern of the Huperzine B crystal form A of Example 4;

FIG. 5 is an X-ray powder diffraction pattern of the Huperzine B crystal form A of Example 5;

FIG. 6 is an X-ray powder diffraction pattern of the Huperzine B crystal form A of Example 6;

FIG. 7 is a typical infrared spectrum pattern of the Huperzine B crystal form A;

FIG. 8 is a typical Raman spectrum of the Huperzine B crystal form A;

FIG. 9 is an XRPD pattern of the Huperzine B Crystal form B of Comparative Example 1;

FIG. 10 shows a comparative XRPD pattern of the Huperzine B crystal form A of Example 1 and the Huperzine B crystal form B of Comparative Example 1.

### DETAILED DESCRIPTION OF THE INVENTION

After a long-term and in-depth research, the present inventors have prepared and obtained a crystal form of Huperzine B. The crystal form has very low hygroscopicity and good stability, and is therefore suitable for long-term preservation. Based on the above finding, the inventors completed the present invention.

### Crystal form A of Huperzine B

The present invention discloses a crystal form A of Huperzine B, and the X-ray diffraction pattern of which, obtained using Cu-Ka radiation, has absorption peaks with 2θ angular expressed in degrees at: 9.56, 12.08, 13.26, 13.90, 14.12, 14.88, 15.18, 16.00, 16.86, 18.45, 19.12, 19.84, 22.41, 22.97,23.42, 24.79, 25.39, 25.81, 26.01, 26.34, 27.65, 28.04, 28.44, 28.78, 29.86, 30.51, 31.22, 33.22, 34.94, 37.09, and 38.64. The positions of the absorption peaks are changed by about 5% depending on the instruments, but the arrangement and shape of the peaks remain the same.

The infrared spectrum of the crystal form A of Huperzine B, as measured by KBr compression, has absorption peaks at 3323.36 cm⁻¹, 3219.52 cm⁻¹, 3088.83 cm⁻¹, 2994.76 cm⁻¹, 2958.33 cm⁻¹, 2938.33 cm⁻¹, 2913.38 cm⁻¹, 2861.67 cm⁻¹, 2817.87 cm⁻¹, 1666.30 cm⁻¹, 1605.50 cm⁻¹, 1555.70 cm⁻¹, 1453.09 cm⁻¹, 1429.96 cm⁻¹, 1369.99 cm⁻¹, 1346.50 cm⁻¹, 1323.03 cm⁻¹, 1309.51 cm⁻¹, 1283.99 cm⁻¹, 1260.62 cm⁻¹, 1200.47 cm⁻¹, 1180.20 cm⁻¹, 1155.23 cm⁻¹, 1127.13 cm⁻¹, 1099.03 cm⁻¹, 1071.87 cm⁻¹, 1055.99 cm⁻¹, 1046.15 cm⁻¹, 993.81 cm⁻¹, 973.82 cm⁻¹, 958.22 cm⁻¹, 915.72 cm⁻¹, 867.03 cm⁻¹, 855.87 cm⁻¹, 839.87 cm⁻¹, 815.10 cm⁻¹, 783.02 cm⁻¹, 755.87 cm⁻¹, 731.69 cm⁻¹, 686.04 cm⁻¹, 651.90 cm⁻¹, 629.38 cm⁻¹, 568.32 cm⁻¹, 521.58 cm⁻¹, 499.42 cm⁻¹, 487.62 cm⁻¹, 467.39 cm⁻¹, 442.58 cm⁻¹, and 417.99 cm⁻¹. The positions of the absorption peaks are changed by about 2% depending on the instruments, but the arrangement and shape of the peaks remain the same.

The measured Raman spectrum of the crystal form A of Huperzine B has absorption peaks at 2939.42 cm⁻¹, 2909.62 cm⁻¹, 2879.72 cm⁻¹, 2864.57 cm⁻¹, 2820.5 cm⁻¹, 1674.43 cm⁻¹, 1602.88 cm⁻¹, 1553.75 cm⁻¹, 1450.8 cm⁻¹, 1288.9 cm⁻¹, 1262.33 cm⁻¹, 1246.66 cm⁻¹, 719.965 cm⁻1 and 683.93 cm⁻¹.

### Crystal form A of Huperzine B

The present invention also provides a preparation method of crystal form A of HuperzineB, the crystal form can be prepared by methods selected from the group consisting of the following Method I, Method II and Method III:

### Method I:

1) adding the self-made raw material of Huperzine B to the solvent;
2) heating under stirring to dissolve it;
3) cooling down and placing the solution in a wide-mouth glass container to evaporate the solvent to dryness; and
4) drying in vacuum and collecting the resulting solid;
wherein the solvent is selected from methanol, ethanol, n-butanol, 1,4-dioxane, 1,2-dichloroethane, dichloromethane, acetone, water, and combinations thereof.
wherein the volume-mass ratio of solvent to Huperzine B in step 1) is 1:15 to 1:75 (ml: mg); wherein the heating temperature in step 2) is 50° C. to 90° C.

### Method II:

1) adding the self-made raw material of Huperzine B to the solvent;
2) heating under stirring to dissolve it;
3) cooling down to room temperature to precipitate crystals, and keeping the temperature at about 0° C.;
4) filtering, drying in vacuum and collecting the resulting solid;
wherein the solvent is selected from ethanol, n-butanol, 1,4-dioxane, 1,2-dichloroethane, N,N-dimethylformamide, acetone, water, methyl tert-butyl ether, and combinations thereof.
wherein the volume-mass ratio of solvent to Huperzine B in step 1) is 1:15 to 1:90 (ml: mg); wherein the heating temperature in step 2) is 70° C. to 90° C.

### Method III:

1) adding the self-made raw material of Huperzine B to the solvent;
2) heating and stirring and keeping for 72 hours;
3) placing the reaction mixture in a wide-mouth glass container and evaporating the solvent to dryness;
4) drying in vacuum and collecting the resulting solid;
wherein the solvent is selected from the group consisting of toluene, ethyl acetate, methyl tert-butyl ether, and combinations thereof.
wherein the volume-mass ratio of solvent to Huperzine B in step 1) is 1:2 to 1:10 (ml: mg); wherein the heating temperature in step 2) is 40° C. to 60° C.

The crystal form A of Huperzine B, as a butyrylcholinesterase, has a certain inhibitory effect on the activity of AchE in the cerebral cortex and BuchE in the serum of rats, and is used in the preparation of drugs for the treatment of neurodegenerative diseases.

The neurodegenerative diseases include Alzheimer's disease, vascular dementia, mental retardation, schizophrenia and memory disorders.

Compared with the prior art, the main advantages of the present invention lie in that: the study of the crystal form of a drug and the stability of the crystal form play a crucial role in the development of the drug, and the present inventor, through the study, provides a crystal form A of Huperzine B and a method for the preparation thereof. The crystal form A of Huperzine B has friendly physicochemical properties and good stability, and is suitable for preparation on an industrial scale; the method for the preparation of crystal form A is easy to operate, reproducible, and has a high purity of the product; and the crystal form A has stable performance and controllable product quality in the prepared formulations, which provides effective crystal form data to support the clinical development of new drugs. The crystal form of Huperzine B of the present invention has successfully filled the gap of this technology in the existing pharmaceutical field.

The present invention is further described below in conjunction with specific examples. It is to be understood that these examples are intended to illustrate the invention only and not to limit the scope of the invention. The experimental methods in the following examples that do not specify specific conditions are usually based on conventional conditions or conditions recommended by the manufacturer. Unless otherwise specified, percentages and parts are calculated by weight.

The invention is further illustrated by the following examples, but the scope of the invention is not limited thereto.

### Experimental Conditions:

XRPD (X-ray powder diffraction): the XRPD patterns in the present invention were detected by a Bruker Advance X-ray diffractometer with a 2θ angular scan from 5 degrees to 45 degrees, Cu-Ka radiation.

IR (Infrared Spectroscopy): the IR spectrums in the present invention were detected by a Bruker Tenso2 27 Infrared Absorption Spectrometer with a detection range of 4000-350 wave numbers.

Raman (Raman Spectroscopy): The Raman spectrum in the present invention was detected by a DXR Micro Raman Spectrometer with a detection range of 3500-50 cm⁻¹ Raman shift.

The conditions and methods for detecting the relevant substances involved in the present invention are high performance liquid chromatography determination.

Chromatographic conditions: octadecylsilane-bonded silica gel as stationary phase (Waters Symmetry C18 column, 250×4.6 mm, 5 μm), 0.02 mol/L phosphate buffer solution, phosphoric acid adjusted to pH 2.5 as mobile phase A, acetonitrile as mobile phase B, A: B=90:10, isocratic elution; flow rate: 1 ml/min; column temperature: 25° C.; PAD detector; detection wavelength: 310 nm.

### General Method: Preparation of Huperzine B API

The raw material of Huperzine B used in this study is obtained by extracting and refining of Huperziaserrata (Thunb.) Trev., and the detailed process is as follows:

Extraction: 100 Kg of Huperziaserrata (Thunb.) Trev. was crushed and added into an extraction tank, and extracted with 20 times amount of 1.5% tartaric acid aqueous solution at 45° C. with dynamic stirring for 36 hours;

Concentration: the leachate was concentrated to 100 L;

Extraction: the concentrated solution was adjusted to pH 9 with dilute alkaline ammonia, and extracted with chloroform repeatedly for 4 times. the chloroform extracts were combined, and concentrated and chloroform was recycled, concentrated to 100 L;

Acid counter-extraction: the above solution was repeatedly extracted with 2% citric acid aqueous solution for 3 times;

Decolorization with activated carbon: the acidaqueous extraction solution was diluted 10 times with water, and the amount of activated carbon was 1% of the diluted volume. After stirring for 30 minutes, filtration was carried out to obtain a transparent liquid;

Concentration: the above solution was concentrated to 200 L;

Column chromatography: the above extract was loaded onto a silica gel column, and a gradient elution was carried out using methanol-chloroform eluent to collect methanol-chloroform (25:75) eluent fractions;

The above fractions were combined and concentrated to 500 ml and sent to v vacuum refrigerating machine for lyophilization to obtain 50 g of Huperzine B extract.

Column chromatography: the Huperzine B extract was put into a reactor, dissolved in 2 times volume of chloroform/ethanol mixed solvent (v/v 98:2), and then subjected to silica gel column chromatography, and resolved with chloroform/ethanol mixed solvent (v/v 97:3). When the resolution solution was negative by TLC, the resolution was stopped. The resolution solution was concentrated to dryness under reduced pressure, and then dissolved by adding 10 times volume of chloroform. The chloroform phase was treated with dilute ammonia, dried, and concentrated to obtain a crude product;

Crystallization: the crude product was added into 5 times amount of acetone, refluxed with heat in a water bath for 1 hour, and then filtered while hot. The filtrate was placed to precipitate crystals, drained, and dried at 80° C. in a vacuum oven for 2 hours, to obtain Huperzine B, 1.7 g, purity>96%.

### Example 1 Preparation of Huperzine B crystal form A (Method 1a)

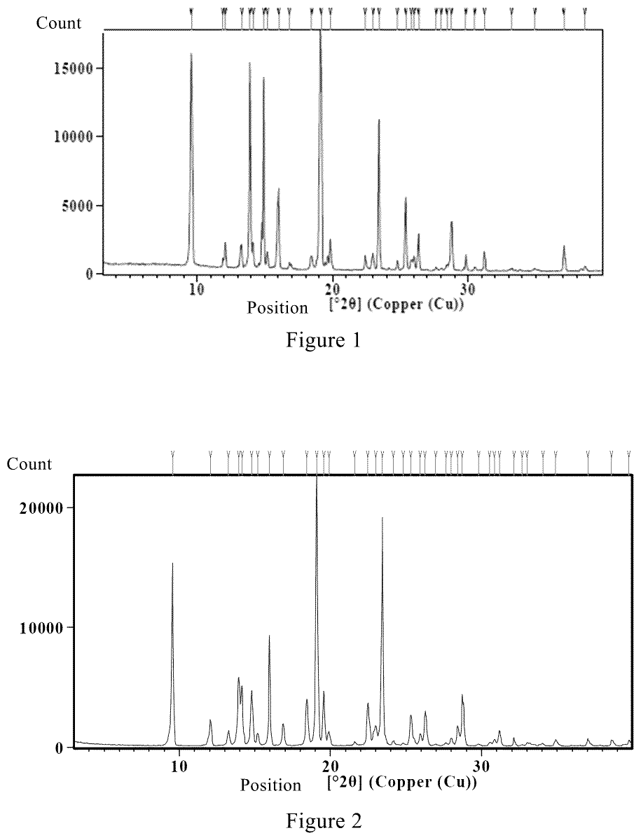

120 mg of the above Huperzine B powder was weighed and added to 3 ml ethanol, heated to 70° C. to dissolve completely, and then cooled down. The solution was placed in a wide-mouth glass container to evaporate the solvent to dryness, dried in vacuum, and collected to obtain 108 mg of crystalline powder, with a yield of 90%. As determined by X-ray powder diffraction, the obtained crystalline powder was crystal form A, the pattern was as shown in FIG. 1, and the specific peaks were as shown in the Table 1 below:

TABLE 1

| 2θ | d(A) | I% |
|---|---|---|
| 9.56 | 9.25 | 88.86 |
| 12.08 | 7.33 | 10.58 |
| 13.26 | 6.68 | 8.06 |
| 13.90 | 6.37 | 85.34 |
| 14.12 | 6.27 | 10.50 |
| 14.88 | 5.95 | 47.59 |
| 15.18 | 5.84 | 6.87 |
| 16.00 | 5.54 | 31.79 |
| 16.86 | 5.26 | 2.28 |
| 18.45 | 4.81 | 5.65 |
| 19.12 | 4.64 | 100.00 |
| 19.84 | 4.48 | 10.85 |
| 22.41 | 3.97 | 6.25 |
| 22.97 | 3.87 | 6.97 |
| 23.42 | 3.80 | 62.84 |
| 24.79 | 3.59 | 3.86 |
| 25.39 | 3.51 | 30.68 |
| 25.81 | 3.45 | 4.24 |
| 26.01 | 3.43 | 5.99 |
| 26.34 | 3.38 | 15.42 |
| 27.65 | 3.23 | 1.35 |
| 28.04 | 3.18 | 0.96 |
| 28.44 | 3.14 | 2.33 |
| 28.78 | 3.10 | 20.29 |
| 29.86 | 2.99 | 5.40 |
| 30.51 | 2.93 | 1.36 |
| 31.22 | 2.86 | 7.34 |
| 33.22 | 2.70 | 0.94 |
| 34.94 | 2.57 | 1.11 |
| 37.09 | 2.42 | 10.57 |
| 38.64 | 2.33 | 1.95 |

### Example 2 Preparation of Huperzine B Crystal form A (Method 1a)

60 mg of the above Huperzine B was weighed and added into 15 ml of dichloromethane, warmed up to 45° C. to dissolve completely, and then cooled down. The solution was placed in a wide-mouth glass container to evaporate the solvent to dryness, dried in vacuum, and collected to obtain a crystalline powder of 28 mg, with a yield of 46.6%. As determined by X-ray powder diffraction, the obtained crystalline powder was crystal form A, and the pattern was shown in FIG. 2.

### Example 3 Preparation of Huperzine B Crystal form A (Method 1b)

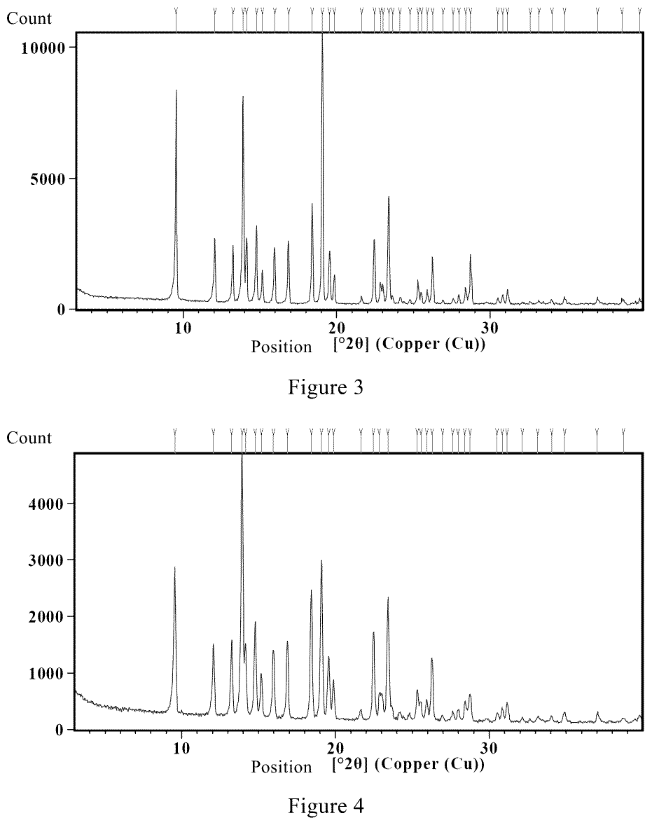

120 mg of the above Huperzine B was weighed and added to 3.5 ml of ethanol, heated to 70° C. to dissolve completely, and then slowly reduced to room temperature to precipitate crystals. When the crystals were precipitated, the reaction solution was further reduced to 0° C. and kept for 1 hour. After filtration, the solid was dried in vacuum to obtain 36 mg of crystalline powder, with a yield of 30%. As determined by X-ray powder diffraction, the obtained crystalline powder was crystal form A, and the pattern was shown in FIG. 3.

### Example 4 Preparation of Huperzine B Crystal form A

60 mg of the above Huperzine B was weighed and added to 2 ml of n-butanol, heated to 80° C. to dissolve completely, then reduced to 50° C. and added with 20 ml of methyl tert-butyl ether. The temperature was reduced to precipitate crystals. After the crystals were precipitated, the reaction solution was further reduced to 0° C. and kept for 1 hour. After filtration, the solids were dried in vacuum to obtain 47 mg of crystalline powder, with a yield of 78.3%. As determined by X-ray powder diffraction, the obtained crystalline powder was crystal form A, and the pattern was shown in FIG. 4.

### Example 5 Preparation of Huperzine B Crystal form A (Method 1b)

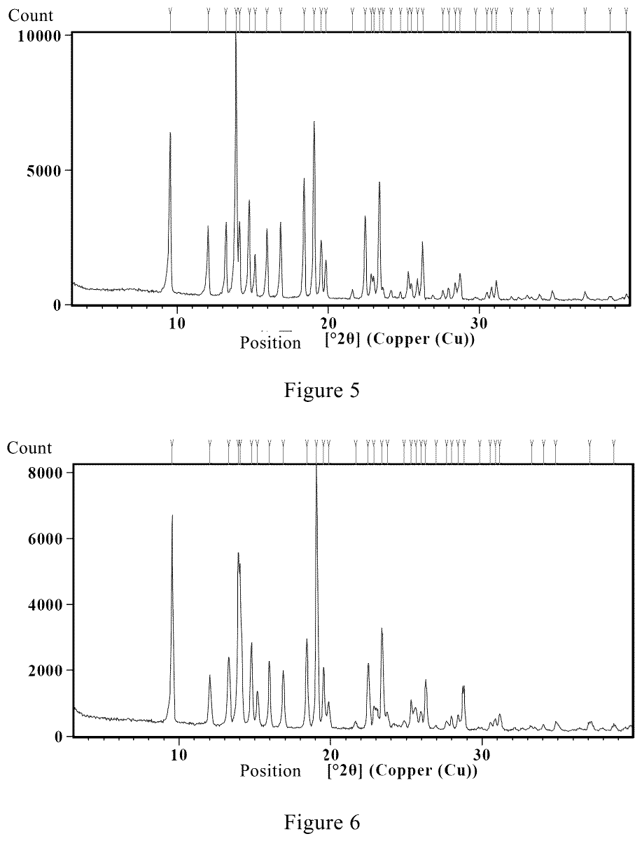

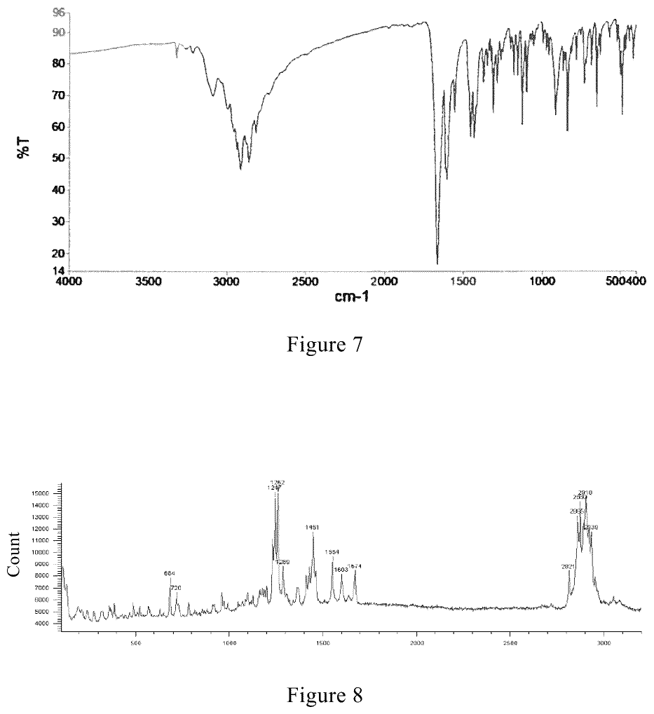

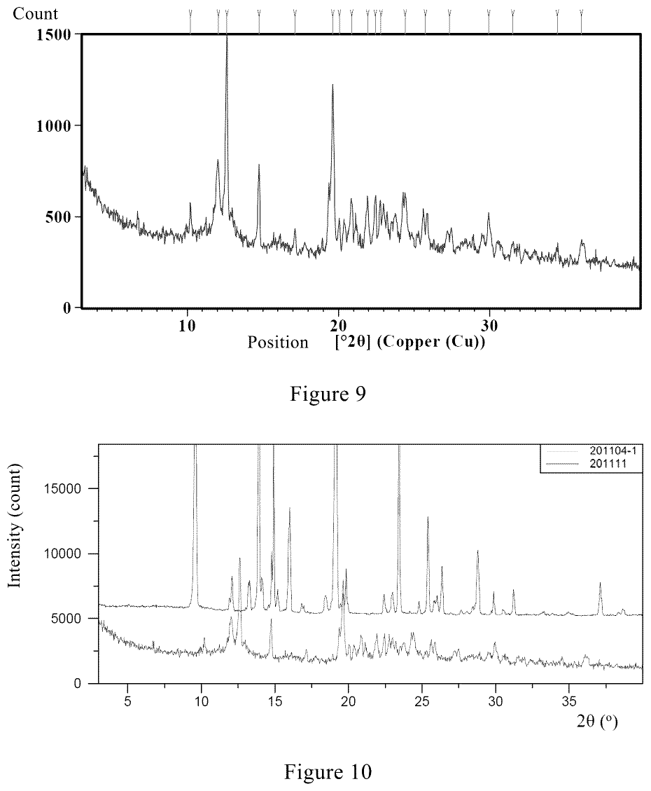

62 mg of the above Huperzine B was weighed and added to 5 ml of 1,4-dioxane, heated to 90° C. to dissolve completely, and cooled down to precipitate crystals. After the crystals were precipitated, the reaction solution was further reduced to 0° C. and kept for 1 hour. After filtration, the solids were dried in vacuum to obtain 39 mg of crystalline powder with a yield of 62.9%. As determined by X-ray powder diffraction, the obtained crystalline powder was crystal form A, the pattern was shown in FIG. 5.

### Example 6 Preparation of Huperzine B Crystal form A

60 mg of the above Huperzine B was weighed and added to 10ml of toluene, heated to 50° C., kept and stirred for 72 hours, and then cooled down. The solution was placed in a wide-mouth glass container to evaporate the solvent to dryness, dried in vacuum, and collected to obtain 36 mg of crystalline powder, with a yield of 60%. As determined by X-ray powder diffraction, the obtained crystalline powder was crystal form A, and the pattern was shown in FIG. 6.

### Comparative Example 1 Preparation of Huperzine B Crystal form B

60 mg of the above Huperzine B was weighed and added into 5 ml of ethanol, and heated to 70-75° C. to dissolve completely. 3 ml of purified water was added dropwise into the solution, and a large amount of solid precipitated. After filtration, the solid was dried in vacuum, and collected to obtain 32 mg of crystalline powder, with a yield of 53.3%. As determined by X-ray powder diffraction, the obtained crystalline powder was crystal form B, and the pattern was shown in FIG. 10. The specific peaks were shown in Table 2 below:

TABLE 2

| 2θ | d(A) | I% |
|---|---|---|
| 10.20 | 8.68 | 17.85 |
| 12.04 | 7.35 | 39.00 |
| 12.62 | 7.01 | 100.00 |
| 14.74 | 6.01 | 39.62 |
| 17.12 | 5.18 | 10.35 |
| 19.62 | 4.52 | 81.41 |
| 20.06 | 4.43 | 16.63 |
| 20.88 | 4.25 | 24.49 |
| 21.93 | 4.05 | 27.53 |
| 22.44 | 3.96 | 27.77 |
| 22.79 | 3.90 | 17.96 |
| 24.42 | 3.65 | 28.88 |
| 25.75 | 3.46 | 15.25 |
| 27.34 | 3.26 | 10.41 |
| 29.94 | 2.98 | 20.52 |
| 31.53 | 2.84 | 7.28 |
| 34.48 | 2.60 | 5.23 |
| 36.06 | 2.49 | 9.84 |

### Test Example 1 Hygroscopicity Test of Huperzine B Crystal form A

The test was carried out with reference to the requirements of hygroscopicity test in the General Technical Requirements of Part IV of the Chinese Pharmacopoeia 2020 Edition.

Methods: A dry stoppered glass weighing flask (outer diameter 50 mm, height 15 mm) was placed in a suitable 25° C.±1° C. constant temperature desiccator (with ammonium chloride or ammonium sulfate saturated solution placed in the lower part) or an artificial climate chamber (with a set temperature of 25° C.±1° C. and a relative humidity of 80%±2%) prior to the test, and the weight (m₁) was weighed precisely.

An appropriate amount of the test sample (Huperzine B crystal form A prepared in Example 1 and Huperzine B crystal form B prepared in Example 7) was taken respectively, and spread flat in the above weighing flask. The thickness of the test sample was generally 1 mm, and the weight (m₂) was weighed precisely. The weighing flask was left open, and was placed in the above constant temperature and humidity conditions together with the cap of the flask for 24 hours.

The weighing flask was closed with the cap and weighed precisely (m₃).

$$Percent age of weight gain = \frac{m 3 - m 2}{m 2 - m 1} \times 100 \%$$

The test results are shown in Table 4.

| Crystalline | Amount of test | Percentage of | |
|---|---|---|---|
| type | sample (g) | weight gain (%) | Conclusion: |
| Crystal | 0.9507 | 0.05 | No or almost no |
| form A | | | hygroscopicity |
| Crystal | 1.0011 | 2.88 | hygroscopicity |
| form B | | | |

The experimental results showed that Huperzine B crystal form A obtained by the present invention has low hygroscopicity and is therefore more suitable for long-term preservation than Huperzine B crystal form B.

### Test Example 2 Examination of the Stability of Crystals

### 1. High Temperature Test

Methods: The test samples (Huperzine B crystal form A prepared in Example 1 and Huperzine B crystal form B prepared in Example 7), respectively, were placed in an open, clean glassware and placed in a constant temperature desiccator at 60° C. The samples were taken and tested on day 5 and day 10, respectively, and the results were compared with those on day 0.The results are shown in Table 5.

Examination item

| Time | | | relevant | |
|---|---|---|---|---|
| (days) | Huperzine B | Appearance | substances | Content |
| 0 | Crystal form | Off-white | 0.12 | 100.1% |
| | A | powder | | |
| | Crystal form | Off-white | 0.18 | 99.8% |
| | B | powder | | |
| 5 | Crystal form | Off-white | 0.19 | 100.2% |
| | A | powder | | |
| | Crystal form | Off-white | 0.29 | 99.5% |
| | B | powder | | |
| 10 | Crystal form | Off-white | 0.21 | 99.7% |
| | A | powder | | |
| | Crystal form | Off-white | 0.41 | 98.9% |
| | B | powder | | |

### 2. High Humidity Test

Methods: The test samples (Huperzine B crystal form A prepared in Example 1 and Huperzine B crystal form B prepared in Example 7), respectively, were evenly distributedinan open petri dish with a thickness of ≤5 mm, and placed in a constant temperature and humidity incubator at room temperature (around 25° C.) with a relative humidity of 75±5%. The samples were taken and tested on day 5 and day 10, respectively, and the results were compared with that of day 0. The results are shown in Table 6.

| | | Examination item | | |
|---|---|---|---|---|
| Time (days) | Huperzine B | Appearance | Moisture | Content |
| 0 | Crystal form | Off-white powder | 6.93% | 100.1% |
| | A | | | |
| | Crystal form | Off-white powder | 6.13% | 99.8% |
| | B | | | |
| 5 | Crystal form | Off-white powder | 6.95% | 100.2% |
| | A | | | |
| | Crystal form | Off-white powder | 7.13% | 99.7% |
| | B | | | |
| 10 | Crystal form | Off-white powder | 6.98% | 99.9% |
| | A | | | |
| | Crystal form | Off-white powder | 7.53% | 99.6% |
| | B | | | |

### 3. Strong Light Irradiation Test

Methods: The test samples (Huperzine B crystal form A prepared in Example 1 and Huperzine B crystal form B prepared in Example 7), respectively, were evenly distributed in an open petri dish with a thickness of ≤5 mm. The distance was adjusted so that the light intensity was 4,500±500 Lx. The samples were taken and tested on day 5 and day 10, respectively, and the results were compared with that of day 0. The results are shown in Table 7:

| | | Examination item | | |
|---|---|---|---|---|
| Time | | | relevant | |
| (days) | Huperzine B | Appearance | substances | Content |
| 0 | Crystal form A | Off-white powder | 0.12 | 100.1% |
| | Crystal form B | Off-white powder | 0.18 | 99.8% |
| 5 | Crystal form A | Off-white powder | 0.22 | 99.8% |
| | Crystal form B | Off-white powder | 0.32 | 99.5% |
| 10 | Crystal form A | Off-white powder | 0.28 | 98.9% |
| | Crystal form B | Off-white powder | 0.49 | 98.6% |

The results showed that the compounds of the present invention have better stability than another crystal form B of Huperzine B in high temperature, high humidity and strong light irradiation tests.

### Test Example 3 Study Oncholinesterase Inhibitory Activity of Crystal form A of Huperzine B in vivo

Huperzine B is a highly selective acetylcholinesterase inhibitor (AChEI) for AChE in the brain, which is extracted and purified from the Chinese herb Huperziaserrata (Thunb.) Trev. Acetylcholinesterase inhibitor is one of the few effective drugs approved by the FDA for the treatment of Alzheimer's disease.

Experimental animals: SD rats, clean grade, male, body weight 180-200 g, provided by Shanghai SLAC Laboratory Animal Co., Ltd, free to drink and ingest during the experimental period, indoor temperature 22-24° C., humidity 45-70%.

Test Subject: Huperzine B (hupB).

Main reagents: S-Butyrylthiocholine iodide, S-Acetylthiocholine iodide, Kaumas Brilliant Blue, sodium hydroxide, ethyl carbamate (urethane), phosphoric acid, sodium chloride, sodium bicarbonate, potassium dihydrogen phosphate, ethanol, sodium dodecyl sulfate, 5,5′-Dithiobis-(2-nitrobenzoic acid) (DTNB), hydrochloric acid; the above chemical reagents were purchased from Sinopharm Chemical Reagent Co., Ltd. Physiological saline, product of Shanghai Bangjing Industry Co., Ltd; bovine serum albumin, purchased from Meilun Biologicals.

Experimental grouping and dose setting: SD rats were randomly grouped, i.e., blank control group NS, and subject hupB groups (0.48 mg/kg, 0.24 mg/kg and 0.12 mg/kg) with 11 animals in each group; 20 mL/kg of drug administered by gavage.

Acetylcholinesterase activity was calculated using the equation:

[ΔA₄₁₂/13600×(0.1/1000)×(4/0.1)×(1/8)×10⁹]/[10×protein content×(1/1000)] (nmol/min/mg protein)

Acetylcholinesterase activity rate was calculated by the equation:

$$[ \frac{Sample AChEactivity}{ControlAChEactivity} - 1 ] \times 100 \%$$

The equation for calculating the activity and the activity rate of “butyrylcholinesterase” was the same as that for “acetylcholinesterase”.

Statistical analysis: SPSS 18 statistical software and EXCEL were applied. Measurement data were expressed as “mean±standard deviation (x±s)”, t-test was used for comparison between two groups, and LSD method was used for pairwise comparison between groups when there was homogeneity of variance, and Dunnett's method was used for analysis when there was heterogeneity of variance. P<0.05 indicates significant differences.

### Results

(1) The effect of Huperzine B on the dose-response relationship of AchE activity in the hippocampus of rats (x±s)

hippocampus

| | Dose | AchE activity | AchE activity |
|---|---|---|---|
| Group | (mg/kg) | (nmol/min/mg protein) | inhibition rate(%) |
| NS | — | 91.9 ± 9.6 | — |
| hupB | 0.48 | 70.3 ± 8.56* | 23.5 ± 9.3 |
| | 0.24 | 85.5 ± 11.3 | 7.0 ± 12.3 |
| | 0.12 | 93.8 ± 24.9 | −2.1 ± 27.1 |

Note:
Compared to NS group:
*P < 0.05

(2) The effect of Huperzine B on the dose-response relationship of AchE activity in the cortex of rat (x±s)

Cortex

| | Dose | AchE activity | AchE activity |
|---|---|---|---|
| Group | (mg/kg) | (nmol/min/mg protein) | inhibition rate(%) |
| NS | − | 103.8 ± 26.4 | — |
| hupB | 0.48 | 73.9 ± 11.0* | 28.9 ± 10.6 |
| | 0.24 | 62.1 ± 8.00* | 40.3 ± 7.7 |
| | 0.12 | 97.6 ± 46.9 | 6.2 ± 45.1 |

Note:
Compared to NS group:
*P < 0.05

(3) The effect of Huperzine B on the dose-response relationship of AchE activity in the striatum of rat (x±s)

Striatum

| | Dose | AchE activity | AchE activity |
|---|---|---|---|
| Group | (mg/kg) | (nmol/min/mg protein) | inhibition rate(%) |
| NS | — | 220 ± 49 | — |
| hupB | 0.48 | 133 ± 74* | 39.2 ± 26.9 |
| | 0.24 | 165 ± 71* | 25.0 ± 16.0 |
| | 0.12 | 247 ± 81 | −12.3 ± 25.6 |

Note:
Compared to NS group:
*P < 0.05

(2) The effect of Huperzine B on the dose-response relationship of AchE activity in the serum of rat (x±s)

Serum

| | Dose | AchE activity | AchE activity |
|---|---|---|---|
| Group | (mg/kg) | (nmol/min/mg protein) | inhibition rate(%) |
| NS | — | 22.8 ± 1.6 | — |
| hupB | 0.48 | 15.4 ± 3.2* | 32.4 ± 14.2 |
| | 0.24 | 17.9 ± 3.5 | 21.4 ± 15.1 |
| | 0.12 | 24.8 ± 4.5 | −8.6 ± 19.9 |

Note:
Compared to NS group:
*P < 0.05

(5) The effect of Huperzine B on BuchE activity in the serum of rats (x±s)

Serum

| | Dose | Serum BuchE | Inhibition rate |
|---|---|---|---|
| Group | (mg/kg) | (nmol/min/mg protein) | (%) |
| NS | — | 6.89 ± 1.83 | — |
| hupB | 0.48 | 4.88 ± 0.94* | 29.2 ± 13.7 |
| | 0.24 | 5.53 ± 1.94 | 19.9 ± 28.1 |
| | 0.12 | 6.83 ± 3.21 | 1.0 ± 46.5 |

Note:
Compared to NS group:
*P < 0.05

Conclusion: Huperzine B crystal form A can significantly reduce AchE activity in rat cerebral cortex, and have certain inhibitory effect on AchE activity in the rat brain serum, and have certain inhibitory effect on BuchE activity in the rat serum. Therefore, Huperzine B crystal form A, as a cholinesterase inhibitor, has certain inhibitory effect on both acetylcholinesterase and butyrylcholinesterase, and the inhibitory effect on butyrylcholinesterase is stronger. The above experimental data are statistically significant.

All documents referred to in the present invention are incorporated by reference herein as if each document is individually incorporated by reference. Further, it should be understood that upon reading the above teaching of the present invention, various changes or modifications may be made to the present invention by those skilled in the art, and those equivalents also fall within the scope defined by the appended claims of the present application.

1. A crystal form A of Huperzine B, wherein the X-ray diffraction pattern of the crystal form A has characteristic absorption peaks at the following 2θ angular: 9.56±0.2, 13.90±0.2, 14.88±0.2, 16.00±0.2, 25.39±0.2 and 28.78±0.2.

2. The crystal form of claim 1, wherein the X-ray diffraction pattern of the crystal form further comprises one or more characteristic absorption peaks at 2θ angular selected from the group consisting of: 12.08±0.2, 13.26±0.2, 14.12±0.2, 15.18±0.2, 16.86±0.2, 18.45±0.2, 19.12±0.2, 19.84±0.2, 22.41±0.2, 22.97±0.2, 23.42±0.2, 24.79±0.2, 25.81±0.2, 26.01±0.2, 26.34±0.2, 27.65=0.2, 28.04±0.2, 28.44±0.2, 29.86±0.2, 30.51±0.2, 31.22±0.2, 33.22±0.2, 34.94±0.2, 37.09±0.2 and 38.64=0.2.

3. The crystal form of claim 1, wherein the infrared spectrum of the crystal form, as measured by KBr compression, has one or more characteristic absorption peaks selected from the group consisting of: 3323.36 cm⁻¹, 3219.52 cm⁻¹, 3088.83 cm⁻¹, 2994.76 cm⁻¹, 2958.33 cm⁻¹, 2938.33 cm⁻¹, 2913.38 cm⁻¹, 2861.67 cm⁻¹, 2817.87 cm⁻¹, 1666.30 cm⁻¹, 1605.50 cm⁻¹, 1555.70 cm⁻¹, 1453.09 cm⁻¹, 1429.96 cm⁻¹, 1369.99 cm⁻¹, 1346.50 cm⁻¹, 1323.03 cm⁻¹, 1309.51 cm⁻¹, 1283.99 cm⁻¹, 1260.62 cm⁻¹, 1200.47 cm⁻¹, 1180.20 cm⁻¹, 1155.23 cm⁻¹, 1127.13 cm⁻¹, 1099.03 cm⁻¹, 1071.87 cm⁻¹, 1055.99 cm⁻¹, 1046.15 cm⁻¹, 993.81 cm⁻¹, 973.82 cm⁻¹, 958.22 cm⁻¹, 915.72 cm⁻¹, 867.03 cm⁻¹, 855.87 cm⁻¹, 839.87 cm⁻¹, 815.10 cm⁻¹, 783.02 cm⁻¹, 755.87 cm⁻¹, 731.69 cm⁻¹, 686.04 cm⁻¹, 651.90 cm⁻¹, 629.38 cm⁻¹, 568.32 cm⁻¹, 521.58 cm⁻¹, 499.42 cm⁻¹, 487.62 cm⁻¹, 467.39 cm⁻¹, 442.58 cm⁻¹, and 417.99 cm⁻¹.

4. The crystal form of claim 1, wherein the Raman spectrum of the crystal form has one or more characteristic absorption peaks selected from the group consisting of: the absorption peaks were found at 2939.42 cm⁻¹, 2909.62 cm⁻¹, 2879.72 cm⁻¹, 2864.57 cm⁻¹, 2820.5 cm⁻¹, 1674.43 cm⁻¹, 1602.88 cm⁻¹, 1553.75 cm⁻¹, 1450.8 cm⁻¹, 1288.9 cm⁻¹, 1262.33 cm⁻¹, 1246.66 cm⁻¹, 719.965 cm⁻¹ and 683.93 cm⁻¹.

5. A preparation method of the crystal form of claim 1, wherein the method comprises the steps of:
1) dissolving Huperzine B in a first solvent under the heating conditions to obtain a first solution of Huperzine B;
2) performing following step 2a) or 2b):
2a) cooling down the first solution of Huperzine Band slowly evaporating the solvent to dryness;
2b) cooling down to room temperature to precipitate crystals and standing at about 0° C.;
3. drying the product obtained in step 2) in vacuum and collecting the resulting solid to obtain the crystal form.

6. The preparation method of claim 5, wherein when step 2a) is employed, in step 1), the first solvent is selected from the group consisting of: methanol, ethanol, n-butanol, 1,4-dioxane, 1,2-dichloroethane, dichloromethane, acetone, water, and combinations thereof.

7. The preparation method of claim 5, wherein when step 2a) is employed, in step 1), the ratio of solvent to Huperzine B is 1: 15-75 (ml: mg); and/or
in step 1), the heating temperature is 50° C.-90° C.

8. The preparation method of claim 5, wherein when step 2b) is employed, the first solvent is selected from the group consisting of: ethanol, n-butanol, 1,4-dioxane, 1,2-dichloroethane, N,N-dimethylformamide, acetone, water, methyl tert-butyl ether, and combinations thereof.

9. The preparation method of claim 5, wherein when step 2b) is employed, in step 1), the ratio of solvent to Huperzine B is 1:15-90 (ml: mg); and/or
in step 1), the heating temperature is 70° C.-90° C.

10. A preparation method of the crystal form of claim 1, wherein the method comprises the steps of:
i) adding Huperzine B to a second solvent, heating and stirring and keeping for 48-96 hours to obtain a reaction mixture;
ii) placing the reaction mixture in an open container and evaporating the solvent to dryness at room temperature;
iii) drying in vacuum and collecting the resulting solid to obtain the crystal form.

11. The preparation method of claim 10, wherein the second solvent is selected from the group consisting of: toluene, ethyl acetate, methyl tert-butyl ether, and combinations thereof.

12. The preparation method of claim 10, wherein in step i), the ratio of solvent to Huperzine B is 1: 2-10 (ml: mg); and/or
in step ii), the heating temperature is 40° C.-60° C.
